(19) United States
(12) Patent Application Publication
(10) Pub. No.: US 2010/0035248 A1
(43) Pub. Date: Feb. 11, 2010

(54) SURFACE-BASED NUCLEIC ACID ASSAYS EMPLOYING MORPHOLINOS

(76) Inventors: Rastislav Levicky, Irvington, NY (US); Napoleon Tercero, New York, NY (US); Ping Gong, New York, NY (US); Kenneth Shepard, Ossining, NY (US)

Correspondence Address: SMITH, GAMBRELL & RUSSELL, SUITE 3100, PROMENADE II, 1230 PEACHTREE STREET, N.E., ATLANTA, GA, 30309-3592, US

(21) Appl. No.: 12/139,477

(22) Filed: Jun. 15, 2008

Related U.S. Application Data
(60) Provisional application No. 60/944,113, filed on Jun. 15, 2007.

(51) Int. Cl. C12Q 1/68; C12M 1/34

(52) U.S. Cl. 435/6; 435/287.2

(57) ABSTRACT

The sequence determination, detection, and quantification of nucleic acid molecules through sequence-specific binding (hybridization) on a solid support, specifically when Morpholinos are used as the surface-immobilized probe species in surface-based nucleic acid assays, and the assays as disclosed herein.

### STATEMENT OF RELATED APPLICATIONS

This application claims the benefit of U.S. Provisional Patent Application No. 60/944,113, filed 15 Jun. 2007 and which is incorporated herein by this reference in its entirety.

### BACKGROUND OF THE INVENTION

1. Technical Field

This invention relates generally to the determination and interpretation of information contained in the base sequence of nucleic acid molecules through sequence-specific binding (hybridization) on a solid support.

2. Prior Art

Clinical, research, forensic, pharmaceutical, environmental monitoring, and medical applications require analysis of nucleic acids. Applications include, but are not limited to, pathogen detection and identification, polymorphism detection, gene expression analysis, genetic sequence identification, genotyping, resequencing, and personalized medicine. For example, analysis of a nucleic acid material can enable a practitioner to identify the origin of the nucleic acid material, such as a virus, bacterium or other prokaryote, or an eukaryotic cell or organism, for applications in forensic analysis or pathogen diagnostics. Detection and measurement of messenger RNA, and of individual-unique DNA polymorphisms (particularly in the portions of genes encoding proteins) enables the practitioner to apply genomic and genetic information to the design, screening, and application of agents such as drugs that will affect, prospectively or retrospectively, the physiological state of an animal tissue in a favorable way. Such knowledge can also enable a practitioner, by detecting the levels of gene expression and protein production, to identify the current physiological state of a tissue or an organism and to predict such physiological states in the future, and to learn the function of genes.

One class of technologies used for this purpose is surface-based nucleic acid assays, as manifested in devices such as DNA chips and microarrays and nucleic acid biosensors. In use, an array surface is contacted with one or more analytes under conditions that promote sequence-specific, high-affinity binding of the analyte molecules to one or more of the array sites. This technology can identify the sequence of nucleic acids and quantify their amount in a sample. The term “sequence” often in the art refers to the order of nucleic acid bases along a polynucleic acid strand, and the sequence-specific binding often in the art is referred to with the term “hybridization”. Hybridization taking place at a solid-liquid interface often in the art is referred to as “surface hybridization”, “solid-phase hybridization”, or “heterogeneous hybridization”. The term “surface hybridization” will be used herein.

In these assays, hybridization takes place on a solid support, and most often, hybridization is realized by modifying the solid support with a single-stranded nucleic acid (typically DNA) that is used to bind nucleic acids from solution according to known base-pairing rules. The immobilized strands are often referred to with the term “probes” while strands in solution are often referred to with the term “targets”. Arrays of immobilized DNA probes can be used to perform highly parallel nucleic acid hybridization assays. Generally, in such surface hybridization assays, labeled single- and/or double-stranded nucleic acid target is hybridized to complementary single-stranded nucleic acid probe sites. The complementary nucleic acid probe binds the labeled target and the presence of the target polynucleotide of interest is detected. In this manner, the identity and amounts of sequences of interest in a sample can be measured.

Surface hybridization is an extensively used technique in biomolecular diagnostics, from gene expression and genotyping to identification of forensic specimens, pathogen detection, ecological studies, and other applications requiring interpretation of genomic or genetic material. Factors that influence the molecular processes underlying surface hybridization are highly complex, reflecting interplay of organization and conformation (including secondary structure) of probe and target species, analyte mass transport, competitive reaction kinetics, and processing steps (e.g. washing). This complexity confounds practice of surface hybridization both in terms of developing experimental protocols and in terms of interpretation of results. These challenges are manifested in discrepancies between results from surface hybridization methods and those from other techniques, as well as between different commercial platforms based on surface hybridization. There is thus great urgency to reduce the complexity of surface hybridization measurements and to place the technology as a whole on a more robust fundamental footing.

The diagnostic power of surface-based assays stems in large part from their ability to monitor many (typically thousands and up to a million) probe-target hybridization reactions in parallel, in a single experiment. Each probe sequence is immobilized at a particular location on the surface, and at the end of the assay the amount of complementary target binding at that point is measured to determine whether, and to what amount, that particular target sequence was present in the sample. There currently does not exist a way of monitoring such large numbers of hybridization reactions in parallel that does not rely on surface hybridization in some way. Generally speaking, surface hybridization allows ready separation of the product (probe-target hybrid), which is attached to a solid phase (substrate), from the sample which is in a liquid state. This assists the subsequent characterization of the probe-target hybridized product. For a microarray, for example, the extent of each of the multitude of reactions that took place on its surface is obtained simply by washing of the array (to remove noncomplementary targets) followed by imaging of the array, typically with a fluorescence array scanner. The intensities of fluorescence at the different microarray spots can then be interpreted in terms of the amounts of bound target sequences, which in turn characterize the sequence composition of the liquid sample.

In addition, a number of modeling and theoretical efforts address the physical principles of surface hybridization based on DNA probes. Models were developed that consider the kinetics of heterogeneous hybridization, and of kinetic perturbations due to washing steps employed in microarray hybridization protocols. Complementing these modeling reports, experimentalists have continued to study surface hybridization processes involving DNA probes under controlled conditions. Such individual studies generally focus on a select range of parameters and have firmly established certain trends, e.g. suppression of hybridization at high probe surface coverages. However, a comprehensive consensus on how probe and target characteristics (length, interactions with the solid support, immobilization geometry, secondary structure, sequence mismatch), assay conditions (e.g. salt conditions, temperature, duration), and processing steps (e.g. washing) impact surface hybridization measurements has yet to emerge.

In known DNA probes assays, the DNA probe layer represents a tremendous concentration of negative, immobilized charge. An incoming target, which is likewise negatively charged, must penetrate this repulsive barrier in order to successfully hybridize. To make this possible it is necessary to employ high salt concentrations S about 1M in order to screen the electrostatic repulsions and lower the barrier to hybridization. High S conditions, however, also have detrimental consequences. The sequence stringency of hybridization is compromised, so that a significant fraction of sample strands that bind are only partially matched to the probes. In order to remove these cross-hybridized sequences and develop a higher contrast for readout, the hybridized surface must be washed with buffer of a lower ionic strength; e.g. S ˜0.1 M or less. The importance of the washing step is underscored by analyses of microarray data indicating that the contrast in commercial assays is dominated, in fact, by the kinetics of the washing step. Experiments confirm that washing has a pronounced effect, but also that sequence-dependent variations in kinetics and approach to assay equilibrium are responsible for intensity variations between microarray spots.

Moreover, under high salt concentration cross-hybridization of background (i.e. partially complementary) nucleic acids with the probes can saturate the surface. The saturation stalls progress of hybridization because, with many probes being involved in complexes with partial complements, they are not immediately available to react with fully complementary targets that arrive later. This effect is symptomatic of all competitive assays where thousands of sample sequences, some of them closely related, compete for the probes. The diagnostic result is thus convoluted with the kinetics of both cross-hybridization and washing, making it highly susceptible to variations in protocols between individuals or laboratories.

Another drawback of high S diagnostics is that elevated salt stabilizes secondary structure in sample strands. Secondary structure in target species is well known to influence rates and yields of hybridization; indeed, these dependencies can be deliberately exploited to analyze target folding. It is important to recognize that secondary structure is a ubiquitous phenomenon. Even when sample solutions are thermally denatured prior to hybridization, at the lower temperatures used for the assay the sample reanneals in parallel with target-probe binding, biasing the surface hybridization in an unpredictable, sequence-dependent fashion. Lastly, another consequence of high S conditions is to screen the strength of electrostatic interactions between hybridized targets and the underlying solid support. This screening is a disadvantage if one desires to use such interactions for assay readout or for control of the hybridization reaction.

If the electrostatic barrier presented by the probe layer to target hybridization were removed, then low salt assay conditions could be used. The only way to truly eliminate the electrostatic barrier is by using uncharged probes. The prospective advantages are enormous as removal of this barrier would allow suppression of cross-hybridization, washing, and sample secondary structure effects that comprise key challenges in the use of these widely implemented diagnostic assays. In addition, if uncharged probes are used then electrostatic interactions with the solid support would be strictly selective to the targets. This selectivity was shown to enhance electrostatic detection and can have similar benefits for electronic control of hybridization. Each one of these benefits would represent a significant advance and, in this regard, surface hybridization diagnostics based on Morpholino probes represent a compelling opportunity.

Accordingly, there is always a need for improved assays for these and other purposes. It is to these needs, among others, that this invention is directed.

### BRIEF SUMMARY OF THE INVENTION

Briefly, according to an embodiment of this invention, Morpholino probes of a known base sequence are used for surface hybridization applications to determine the sequence of nucleic acid molecules from a solution sample. This is distinguishable from the general idea of surface-based nucleic acid assays, whether in an array or other format, in that the probe molecules are specifically of the Morpholino type. The present invention thus comprises an assay with a surface-immobilized Morpholino probe, and not a DNA or PNA probe, and does not require a particular geometric format of the assay.

In summary, the use of uncharged (neutral) Morpholino probes for surface hybridization assays is suitable for this invention for at least the following exemplary reasons:

(1) Ability to carry out surface hybridization assays at low ionic strengths when secondary structure in target species is disrupted. Reduction of target secondary structure is an extremely important consideration for applications as it improves assay kinetics and yields, and simplifies interpretation of assay results.

(2) Elimination of the washing step, if sufficient sequence stringency can be achieved during the hybridization assay itself. Abolishing the washing step greatly simplifies protocols and removes a key source of variability.

(3) Implementation of electronic washing techniques. Electronic washing, which refers to the use of surface electric fields to displace less than fully complementary targets from a Morpholino probe layer, is expected to be more convenient and reproducible than existing fluidic washing methods.

(4) Sensitive, label-free detection of hybridization using electrostatic effects to quantify probe-target binding. Because the Morpholino probes are not charged, detection is highly specific to the targets. This is a great advantage over charged probes such as DNA probes.

Uncharged probe systems such as methylphosphonates and peptide nucleic acids (PNA) are unsuitable for the present invention. Methylphosphonate probes have a rather low binding affinity for complementary nucleic acids, as well as poor water solubility, rendering this type of probe unattractive. PNA probes have the highest binding affinity (that is, most favorable free energy of hybridization per base) for complementary sequences as well as the highest sensitivity to mismatches, both of these exceeding those of DNA probes. It is worth noting that high binding affinity and high sensitivity to base mismatches are expected to go hand-in-hand. High affinity enables use of shorter probe sequences which, in turn, are more perturbed by a single base mismatch since the mismatch represents a larger fraction of the total sequence. Therefore, PNA probes are expected to work best in applications where the primary criterion for success is single-base discrimination using shorter sequences, such as single nucleotide polymorphism (SNP) detection. However, synthetic PNA coupling yields are lower and their solubilities poorer; thus, PNA probes are recommended to not exceed 18 bases in length and to avoid sequences with more than 4 purines in a row. The Morpholino probes used in the present invention, on the other hand, excel in their aqueous solubility and subunit coupling yields. These properties allow virtually any sequence to be synthesized and used in aqueous environments, and Morpholinos up to 31 mers have been made with longer sequences possible. Morpholino probes also possess excellent resistance to enzymatic degradation.

The freedom with regard to length and sequence selection is one significant advantage that Morpholino probe assays of the present invention provide over assays based on PNA probes. Indeed, in many diagnostic applications the crucial criterion is “specificity”. Specificity refers to the ability to uniquely identify the source of a particular target sequence, and requires a probe sequence that is sufficiently long. For example, a hypothetical high affinity 3mer probe would not be very specific, as it will bind target sequences from just about any gene or organism present, as each gene is likely to contain that 3mer base combination within it. It would therefore be useless as a diagnostic of gene expression levels, for example. In comparison, a 20mer sequence is much more specific because it is much less likely to be randomly repeated. It has been predicted that, for a target environment with a complexity comparable to the human genome, a probe should possess approximately 20 to 30 bases. The synthesis of such longer probes benefits from the good coupling yields and solubility of Morpholinos. Interestingly, the very high affinity of probes such as PNA probes is expected to be a detriment when longer sequences are required to enforce specificity. While a high affinity probe is excellent for distinguishing single-base mismatches at shorter lengths, at the longer lengths required for specificity it can lead to increased binding of mismatched sequences unless more severe conditions (for example, higher temperatures) are employed to lower the binding constant.

The Morpholino probe assays of the present invention excel when high specificity is required, a key consideration for many applications of the Morpholino probe assays of the present invention. For example, one such category of applications is identification of viral or bacterial pathogens in medical, environmental, food, or other specimens, where a complex background is present because of multiple sources of genomic material (for example, multiple organisms). Another exemplary category is gene expression measurements, especially for fairly large genomes such as the human genome, where again specificity (for example, to a particular mRNA) is an overriding concern. In such uses, the Morpholino probe assays of the present invention are anticipated to especially excel. Pathogen and gene expression microarrays often use probe lengths of around 70 nucleotides in length.

The present invention discloses use of Morpholino probes for quantification and characterization of genomic information using surface hybridization assay (e.g. microarray) technologies. The principal source of complexity in DNA microarray measurements is the multitude of competing interactions that arise simultaneously: in addition to the desired hybridization between analyte “targets” in solution with complementary “probes” on the microarray features, there are also (1) target-target, (2) probe-probe, and (3) cross-hybridized (i.e. less than 100% complementary) probe-target associations. The Morpholino probe assays of the present invention aim to suppress such interfering molecular interactions, to eliminate nonequilibrium processing steps of target denaturation and array washing, and to bring microarray assays closer to the optimum performance achievable under thermodynamic equilibrium. The Morpholino probe assays of the present invention also provide new capabilities with regard to detection and control of surface hybridization.

Because Morpholino probes, in contrast to DNA probes, bind nucleic acids even under very low salt conditions, surface hybridization microarray assays can be performed under continually denaturing conditions, realized through a combination of low salt and elevated temperature, under which target-target interactions and secondary structure are disrupted. Continually denaturing assays are of great advantage in improving the biological meaning of gene expression data by suppressing unpredictable, sequence-specific biases arising from target-target interactions. However, continually denaturing assays are not possible with DNA microarrays because probe-target interactions also would be disrupted.

Another source of complexity in conventional surface hybridization assays are probe-probe interactions, whose presence suppresses affinity toward target strands. If binding affinities are suppressed, target concentrations have to be that much higher to achieve the same diagnostic signal. Sensitivity can be recovered by separating the probes so that they do not strongly interact with each other, and by controlling the surface chemistry so that the probes do not strongly interact with the surface. The Morpholino probe assays used in the present invention are suitable for such situations.

Another benefit of low salt Morpholino probe assays is that they incorporate a gradually increasing electrostatic stringency into the assay itself, potentially obviating the need for a post-hybridization wash step. Although the probes in Morpholino probe assays of the present invention do not present an electrostatic barrier to incoming target strands, as an assay progresses charge will accumulate at the surface because of target hybridization. The resultant electrostatic repulsions are expected to preferentially melt-off mismatched, less-stably bound targets, thus reducing background from cross-hybridization. In contrast, DNA microarray protocols have to impose stringency in the form of a low salt, post-hybridization wash that adds to the complexity and nonequilibrium character of microarray analysis.

Finally, the lack of probe charge in the Morpholino assays of the present invention, as the probes are nonionic, also (1) enables sensitive label-free detection of target hybridization based on electrostatic transduction, and (2) paves the way for use of electric fields to control probe-target hybridization.

The above features and other features and advantages of this invention will become apparent from the following description of selected preferred embodiments, when read in conjunction with the accompanying drawings.

### BRIEF DESCRIPTION OF THE DRAWINGS

FIGS. 1-16 represent examples and/or results illustrating various preferred embodiments of the present invention.

FIG. 1 (left) shows an end-tethered Morpholino probe film on a gold surface, with a layer of mercaptopropanol (MCP) used to block adsorption of the Morpholino bases. FIG. 1 (right) show IRRAS absorbance spectra before (top or black line) and after (bottom or red line) treatment of the Morpholino film with MCP. Disappearance of the spectral features indicated by arrows indicates successful blocking by MCP of the solid support against nonspecific adsorption of the Morpholino backbone. Other mercapto-alcohol, mercapto-carboxylic acid, or mercapto-oligo (ethylene glycol) blocking agents are expected to be similarly effective. Morpholino sequence: TTTTTTTCCTTCCTTTTTTT. MCP treatment: 1 mM MCP in deionized water for 150 minutes.

FIG. 2A shows a basic scheme for polythiol-based immobilization of Morpholino or other probes on gold electrodes. PMPMS: poly(mercaptopropyl)methyl siloxane. FIG. 2B shows the atomic percentages of phosphorus (P 2p) and nitrogen (N 1s), determined with X-ray photoelectron spectroscopy, before and after immersion of thus immobilized DNA probe layers in hot buffer (95° C. for 1 hr in buffer of 1 M NaCl with 0.015 M sodium citrate at pH 7). The illustrated polythiol method provides highly stable immobilization of probe molecules on gold surfaces, and is similarly expected to benefit immobilization on other noble metals including silver and platinum.

FIG. 3 shows ferrocene-maleimide tags F0 and F2. F0 and F2 are two examples of electroactive tags used in characterization of surface hybridization assays.

FIG. 4 shows a MALDI-TOF mass spectrum of a target oligonucleotide before (5882 or black line) and after (6101 or red line) modification with the F2 tag. Also shown is a schematic structure of the oligo-F2 conjugate.

FIG. 5A inset shows a CV trace of a Morpholino probe layer in 1M phosphate buffer, pH 7, before hybridization (interior or red line) and after hybridization (exterior or black line) to complementary F0-labeled DNA target. Main panel: Background-corrected signal (=hybridized scan minus buffer scan shown in inset). FIG. 5B shows a CV trace from a Morpholino probe film after contact with a solution of a noncomplementary F0-labeled target. FIGS. 5A and 5B demonstrate sequence-specific surface hybridization of a nucleic acid target to a Morpholino probe layer. Probe sequence: 5′ TTT TAA ATT CTG CAA GTG AT-S-S-R 3′; complementary target sequence: 5′ ATC ACT TGC AGA ATT TAA-(F0) 3′; noncomplementary target sequence: 5′ AAA AAA AGG AAG GAA AAA-(F0) 3′. Hybridization was carried out for 32 minutes, at a target concentration of 143 nM.

FIG. 6 shows cyclic voltammograms of F0 and F2 labeled oligonucleotide targets hybridized to Morpholino probes. Background charging currents have been subtracted. The peak separation exhibited by these two tags is approximately 60 mV. Probe and target sequences and hybridization conditions were the same as for FIG. 5.

FIG. 7 shows a time series cyclic voltammetry traces for a Morpholino layer undergoing hybridization to F2-labeled complementary target at a concentration of 140 nM target in 1 M phosphate buffer, pH 7. Background charging currents have been subtracted. FIG. 7 demonstrates capability for real-time monitoring of surface hybridization between electroactively-tagged nucleic acid targets and Morpholino probe layers with cyclic voltammetry.

FIG. 8 shows a comparison of hybridization using DNA and Morpholino probe films at 1 M (left) and 40 mM (right) ionic strengths. Hybridizations were carried out for 32 minutes using F2-labeled targets present at 140 nM concentration in phosphate buffer of the indicated strength. Background charging currents have been subtracted. FIG. 8 demonstrates ability of Morpholino probes to undergo surface hybridization to nucleic acid targets under low salt conditions where DNA probes do not hybridize well. Probe sequence: 5′ TTT TAA ATT CTG CAA GTG AT-S-S-R 3′; complementary target sequence: 5′ ATC ACT TGC AGA ATT TAA-(F2) 3′.

FIG. 9 shows observed changes in differential capacitance of Morpholino probe films after addition of a noncomplementary NC target sequence, followed by addition of the complementary C target. Vertical lines separate the different stages of the experiment. FIG. 9A is in 1M NaCl, pH 7. FIG. 9B is in 8 mM NaCl, pH 7. Capacitance was measured with electrochemical impedance spectroscopy. FIG. 9 demonstrates feasibility of label-free monitoring of surface hybridization between Morpholino probes and nucleic acid targets across a range of ionic strength, using electrostatic transduction. Probe sequence: 5′ TTT TAA ATT CTG CAA GTG AT-S-S-R 3′; complementary target sequence: 5′ ATC ACT TGC AGA ATT TAA 3′; noncomplementary target sequence: 5′ AAA AAA AGG AAG GAA AAA 3′.

FIG. 10A shows the change in the differential capacitance of an MCP-only (“no probe”), DNA probe, and Morpholino probe layers. Change in capacitance ΔC_{d} was monitored vs time t for the various cases following addition of ferrocene-labeled complementary target at t=22 min (vertical dashed line). FIGS. 10B-D show forward CV traces taken before (t=0) and after (t=82 min) addition of target. FIG. 10B shows MCP-only surface (no probes). FIG. 10C shows DNA probe surface. FIG. 10D shows Morpholino probe surface. Conditions: probe 5′ TTT TAA ATT CTG CAA GTG AT 3′; target 5′ ATC ACT TGC AGA ATT TAA-F2 3′; probe surface coverage: 1.5×10¹³ DNA probes/cm²; 2.4×10¹² Morpholino probes/cm²; 140 nM target; buffer: 1M phosphate pH 7. Conditions used to measure capacitance: DC bias: 75 mV vs Ag/AgCl/3M NaCl; ac: 5 mV rms; frequency: 100 Hz to 100,000 Hz. The MCP-only (“no probe”) surface shown in FIG. 10B is a control for nonspecific adsorption. FIG. 10 demonstrates that surface hybridization between Morpholino probes and nucleic acid targets can be sensitively tracked using label-free capacitive detection, which is an example of an electrostatic transduction method. In contrast, surface hybridization using DNA probes did not provide a clear label-free signal.

FIG. 11A shows the molecular structure of ferrocene tags F2 and FN0 and FIG. 11B shows a synthetic scheme for tag F2.

FIG. 12 shows an example of a two-tag CV scan showing both Morpholino probe (FN0) and target (F2) signatures. Scan rate: 5 V/s. Ionic strength: 1 M phosphate buffer, pH 7. FIG. 12 demonstrates capability to simultaneously track probe and target surface coverages with electroactive tags, used for characterization of surface hybridization between Morpholino probes and nucleic acid targets.

FIG. 13 shows the equilibrium fraction of hybridized probes x for hybridization between DNA probes and complementary DNA targets, as a function of probe coverage σ_{P} and salt concentration S. x is given by the ratio of hybridized target coverage σ_{T} to the total probe coverage σ_{P}, x=σ_{T}/σ_{P}. FIG. 13 shows that surface hybridization between DNA probes and nucleic acid targets is prevented at low ionic strengths S and high probe coverages σ_{P}. Target coverages were measured with CV, at a scan rate of 80 mV/s.

FIG. 14A shows melting curves (heating) for Morpholino-DNA hybrids in solution at the indicated salt concentrations (solution: NaClO₄ salt, pH 7). FIG. 14B shows melting curves under the same conditions but for DNA-DNA hybrids. Sequences tested: probe: TTT TAA ATT CTG CAA GTG AT; target: ATC ACT TGC AGA ATT TAA. FIG. 14 demonstrates that, at low salt conditions, Morpholino-DNA duplexes are more stable than DNA-DNA hybrids. These results are used to select conditions under which target-target associations in solution are disrupted so as to not interfere with probe-target hybridization.

FIG. 15 shows the extent of surface hybridization, x, as a function of probe coverage Sp and concentration of phosphate buffer, at pH 7. S_{T} is coverage of hybridized targets. FIG. 15A is for Morpholino probes, and FIG. 15B for DNA probes. Probe sequence tested: TTT TAA ATT CTG CAA GTG AT. Target: ATC ACT TGC AGA ATT TAA. These data show what conditions of probe coverage and salt concentration are available to surface hybridization assays involving a probe type (Morpholino or DNA) and nucleic acid targets.

FIG. 16 depicts a hybridization series for an intermediate buffer strength of 200 mM phosphate buffer, pH 7, on a Morpholino probe layer blocked with MCP.

### DETAILED DESCRIPTION OF PREFERRED EMBODIMENTS

Embodiments of this invention include a method comprising using a nucleic acid analogue, Morpholinos, as the probe species in surface-based nucleic acid assays (referred to herein simply as Morpholino probe assays). Nucleic acid analogues are synthetic (that is, non biological) molecules that bind nucleic acids in a sequence-specific manner. These nucleic acid analogues can be hybridized with sample nucleic acids to identify base sequence, to quantify the amount of the nucleic acids, or to otherwise manipulate the nucleic acids as afforded by their sequence-specific binding to the analogue molecules.

Morpholinos are synthetic molecules that are the product of a redesign of natural nucleic acid structure. Morpholino molecules can be prepared with different lengths, in a range comparable to synthetic nucleic acids. Morpholinos bind (hybridize) to complementary nucleic acid sequences by standard Watson-Crick base-pairing where adenine pairs with thymine or uracil and guanine with cytosine. Structurally, the difference between Morpholinos and DNA is that while Morpholinos have standard nucleic acid bases, those bases are bound to morpholine rings instead of deoxyribose rings. In addition, the interunit linkages between morpholine rings are through phosphorodiamidate groups instead of phosphates for DNA. Replacement of the anionic phosphates with the uncharged phosphorodiamidate groups eliminates the backbone charge in the usual physiological pH range.

One embodiment includes the use of Morpholino probes in surface hybridization applications. As, compared to nucleic acid probes, Morpholino probes are electrically neutral and structurally different, the thermodynamics of their interaction with RNA or DNA are different from DNA-DNA, RNA-RNA, or DNA-RNA hybridization. For example, the electrostatic repulsion between the hybridizing partners can be reduced and surface hybridization between Morpholino probes and nucleic acid targets can proceed under conditions not suitable with DNA or RNA probes; for example, at lower ionic strengths. This bears important advantages. For example, as ionic strength decreases, the secondary structure in solution target species becomes increasingly disrupted, which can improve the thermodynamics and kinetics of the surface hybridization assay. As one advantage, the disruption of target secondary structure can mitigate its difficult-to-predict impact on the extent of hybridization, making the assay more quantitative and sensitive. This embodiment can include assay protocols as well as methods for interpreting assay results.

This embodiment recognizes that inter- or intramolecular target-target associations (e.g. double-stranded regions in the target molecules) in a sample alter both kinetics and thermodynamics of surface hybridization, and that suppression of such associations is essential to realizing quantitative and accurate surface hybridization assays. Target association can have various origins. For pathogen assays, for example, the samples usually come as double-stranded DNA from a preceding PCR amplification. Even when the sample is not a priori expected to be double-stranded, as when messenger RNA is being measured, samples will in general have significant random complementarity and hence presence of target-target associations. Approach to equilibrium can be remarkably slowed down by presence of secondary structure associations in target species. For example, surface hybridizations involving hairpin structures are known to be several-fold slower compared to hybridization of sequences lacking such self-complementarity. Hybridization rates can decrease at elevated salt concentrations as well as at elevated target concentrations, implicating increased association of target species as being responsible for the slowdown. The slower kinetics due to secondary structure presumably reflect a need for a more complex molecular rearrangement that must occur during formation of a desired, complementary probe-target hybrid.

Accounting for the impact of target secondary structure during interpretation of assay results is difficult, if not impossible, to implement in a general way. Rather, a more viable solution is to suppress, as much as possible, the presence of target-target associations during the assay itself. The conventional approach is to carry out thermal denaturation of the sample (at 90° C. to 100° C.) prior to hybridization, followed by hybridization at lower temperatures (typically 35° C. to 70° C.). The lower hybridization temperature is necessary because maintenance of high temperature during assay would not only suppress sample secondary structure but also prevent probe-target hybridization. Conversely, it is crucial to recognize that hybridization at lower temperatures must thus occur in competition with reannealing of the previously disrupted target-target associations. In the case of hybridization involving immobilized DNA probes up to 30% of the nucleic acid target is known to reanneal in solution and thus to become excluded from the detection. Therefore, even if thermal denaturation is implemented pre-hybridization, thermodynamic and kinetic biasing of the assay will still exist. Moreover, because reannealing of the sample is a kinetic process its impact on probe-target hybridization is unpredictable and will be dependent on details of the assay protocol. Thus, while thermal denaturation helps improve diagnostic outcomes, it falls short of resolving interferences from target secondary structure. The conventional use of elevated temperature to overcome secondary structure in target species is hindered by a lack of thermal discrimination—not only is the sample (target) denatured at high temperatures but also probe-target binding would be destabilized. For this reason elevated temperature cannot be used to realize continually denaturing conditions during an assay.

Under this embodiment Morpholino probe assays offer a solution. Morpholino probes have an uncharged backbone so that their binding to nucleic acids proceeds even at low salt concentration S (in fact, experiments show that stability of Morpholino-DNA hybridization increases at lower S). In contrast, DNA-DNA, DNA-RNA, and RNA-RNA hybrids are strongly destabilized at low S because of electrostatic repulsion between strand backbones, since both strands are charged. Therefore, with Morpholino probes low S conditions can be exploited to provide a selective control that preferentially destabilizes secondary structure in a nucleic acid sample while hybridization to probes can still take place. Moreover, by performing assays at low S selective pressure to maintain the sample in a denatured state is maintained throughout the assay. This is a tremendous advantage over thermal denaturation which is applied pre-hybridization, and which is compromised by competition between sample reannealing and probe-target binding during the assay itself. For the purposes of this invention, low salt is approximately of magnitude of about 10 mM or less (10¹ mM); medium salt is approximately of the magnitude of about 100 mM (10² mM), and high salt is approximately of the magnitude of about 1 M (10³ mM). These are approximate ranges, and they are not intended as a strict definition. However, for surface hybridization assays, the definitions are reasonable in that high salt (˜1M) conditions are often used to hybridize, while medium or low salt (100 mM or lower) conditions are used in the wash step to remove less than perfectly complementary targets from the surface.

It is expected that the different scopes of action of salt concentration S and temperature T can complement each other. Thus, the globally destabilizing influence of elevated T may be set to control the level of cross-hybridization (i.e. tolerance to formation of mismatched probe-target hybrids), while the selective action of S is further exploited to minimize interference from sample secondary structure. The present invention optimizes S and T conditions to exploit these contrasts and to optimize protocols for Morpholino probe microarrays or other technologies based on hybridization of nucleic acid targets in solution to Morpholino probes immobilized on a solid support.

In another embodiment, low salt (i.e. low S) surface hybridization assays based on Morpholino probes can afford better sequence stringency (sequence discrimination) due to electrostatic repulsions between surface-hybridized targets that accumulate as hybridization progresses to remove more weakly bound, less complementary targets. Low salt conditions can thus suppress cross-hybridization. Cross hybridization is of tremendous concern in conventional surface hybridization utilizing DNA probes where it is extensive because high salt concentrations are required to overcome the electrostatic barrier between the negatively charged DNA probe layer and the like charged target strands. The high salt allows binding of partially mismatched strands as well, so that early on the probe sites become saturated with many sites occupied by mismatched target strands. After the hybridization step, low ionic strength washes are needed to remove some of the cross-hybridized sequences. The extensive cross-hybridization during such an assay, however, remains a problem because it blocks the surface to further binding during an assay. Thus, an arriving target, even if fully complementary, has to either displace a sample strand from a probe or wait for it to spontaneously separate before the new arrival can bind. This competition for probe sites greatly slows down the forward progress of hybridization. Indeed, signals from complementary hybridization have been observed to continue increasing even after 72 hours, much longer than the time allowed in typical hybridization assays. This slow progress is highly undesirable because it means that diagnostic protocols must be adhered to very strictly, since reproducibility of data relies on arresting the assay at a particular, kinetically-defined (as opposed to equilibrium) state.

It is useful to compare the above progress of hybridization for a high S assay using DNA probes with the expected scenario for a low salt assay using Morpholinos. Initially, because the Morpholino probes are neutral, there is little barrier to entry of target species into the Morpholino probe layer. Both complementary and, it is expected, closely related (partially complementary) target sequences will hybridize with the Morpholino probes. As hybridization proceeds, however, the Morpholino probe layer acquires charge due to binding of the negative targets. The accumulation of charge will present an increasingly repulsive barrier to further target binding, thus preventing complete saturation of the probe sites. This lack of saturation has been observed at low S. In contrast to the fully saturated scenario that occurs under high S conditions, the availability of unoccupied Morpholino probe sites should lead to a faster evolution toward equilibrium, as targets arriving later in the assay will be able to bind right away if they can form a hybrid that is sufficiently stable. The formation of such more stable (i.e. more complementary) probe-target hybrids would increase the electrostatic energy in the layer, and thus is expected to be accompanied by break down of less stable, cross-hybridized hybrids that may have formed during the early stages of the assay. This electrostatic “exchange” mechanism, in which more stable hybrids progressively replace less stable ones through electrostatic repulsion at a distance, would maintain the availability of unhybridized Morpholino probe sites and also provide greater sequence stringency as the assay progresses, possibly eliminating the need for a post-hybridization wash step.

In a further embodiment of this invention, Morpholino probe assays promise superior performance in exploiting surface electrostatics for influencing or for monitoring target hybridization. In such applications the solid support acts as an electrode through which a static or a dynamic surface electric field is applied or, alternately, through which a change in the interfacial charge organization is sensed. Assays based on Morpholino probes are much more versatile for exploiting surface electrostatics than assays based on DNA probes. Because Morpholino probes are not charged, electrostatic coupling to the solid support is exclusive to the targets; i.e. only targets interact with the surface electric field, which can be static or dynamic. This selectivity proved highly beneficial in amplifying label-free capacitive detection of target binding, enabling label-free sensing of probe-target hybridization under conditions when hybridization to DNA probes failed to yield a clear diagnostic signal. For surface hybridization assays based on Morpholino probes the range and strength of surface electrostatic (i.e. derived from the interaction of charges) interactions can be enhanced by carrying out measurements at low salt concentrations. Moreover, application of surface electric fields should allow tuning of the probe-target interaction; for example, by applying electric fields that are attractive or repulsive to targets so as to adjust the strength of the probe-target binding, and/or by altering the activation barrier to target entry into the Morpholino probe layer. Enhanced capabilities to exploit electric field phenomena in surface hybridization assays also enable advances such as implementation of all electronic washing protocols, for example, that stand to improve assay accuracy, sensitivity, ease-of-interpretation, and ease-of-operation.

The electrostatic methods considered above are based on a different principle than that used in some commercially available DNA microelectrode array systems, in which DNA probes are immobilized in a gel above the electrode, outside the direct reach of the surface electric field. In such commercial systems, the electrode is used to electrochemically generate protons in order to perturb the ionic strength of a special zwitterionic buffer in which the DNA probes are immersed. It is this change in ionic strength, rather than direct interaction with the surface electric field, that is used to control hybridization with target molecules in these commercial systems.

Microarray surface hybridization formats offer a powerful combination of features that make them highly attractive for clinical, environmental, research, and biothreat analyses. These include highly multiplexed detection with potential to interrogate for thousands of pathogens simultaneously; “virus discovery” through sequence homology (partial complementarity based on highly conserved genomic regions) to detect unknown or mutated pathogens; and expansion of function beyond pathogen identification, e.g. to also quantify changes in gene expression of the infected host as an aid in diagnosis. Samples are typically prepared using PCR or isothermal amplification techniques, with the resultant double-stranded DNA product used for analysis. Direct detection of certain abundant types of RNA (e.g. 16S rRNA), or of messenger RNA (mRNA) in gene expression studies, also are well known in the art.

Morpholino probe assays are attractive for microarray applications for several reasons. Morpholinos are the only uncharged probe class readily prepared within the range of synthetic lengths typically used in these applications (˜20 to ˜70 bases). Moreover, the types of samples assayed are invariably rich in secondary structure, making low salt denaturing Morpholino probe assays especially attractive. Finally, the portability and performance improvement afforded by electronic detection and hybridization control, as well as protocol simplifications (e.g. due to elimination of washing steps) should be of especial interest in the clinical and field settings that are key application areas for pathogen as well as environmental microarrays.

Nucleic acid assays based on immobilized Morpholino probes, whether implemented in array or other formats, can find use in surface hybridization assays in which a sample is assayed for the presence, either qualitatively or quantitatively, of one or more target nucleic acid sequences. In general, the probe layer is reacted with a sample suspected of including the target nucleotide sequence and the binding of the target nucleotide sequence is then measured, either by labeling the target prior to or after hybridization, or through a label-free approach. Moreover, to accelerate mass transport of targets to the probe layer, the concentration of sample solutions can be elevated so that the quantity of denatured target sequences of interest is in the 0.1 nM or higher range. In this range, equilibrium is expected to be achieved on relatively short time scales (e.g. overnight).

Morpholino probe assays comprise array surfaces with at least one, and possibly many, hybridization features made up of immobilized hybridization probes, and possibly also various types of control probes. By immobilized is meant that the Morpholino probes are stably associated with the surface of the solid substrate during hybridization. In many cases, the Morpholino probes are covalently bonded to the substrate surface, but they could also be immobilized through physical, non-covalent interactions. The solid supports can be conductive so as to allow control over the surface electric potential, or to measure the electric properties of the surface, in order to influence or monitor the hybridization process. The solid supports also can be of nonconductive materials such as glass, silica, or polymers. The solid supports can be flat and planar (e.g. similar to glass slides), curved (e.g. similar to solid beads, a tube, or a fluidic channel), as well as porous or even gel-type supports whose interstitial spaces are filled with buffer or hybridization solvent.

Morpholino probe arrays may be produced using any convenient protocol. Various methods for forming arrays from pre-formed probes, such as probe deposition using spotting pins or non-contact printing methods, or methods for synthesizing probe molecules directly on the solid support, are generally known in the art and have been practiced widely for DNA probes. Similarly, Morpholino probes could either be synthesized directly on the solid support or substrate to be used in the hybridization reaction, or attached to the substrate after they are made. Suitable substrates may exist, for example, as gels, sheets, tubing, spheres, containers, pads, slices, films, plates, slides, strips, plates, disks, rods, particles, microelectronic chips, beads, etcetera. The substrate can be flat, but may take on alternative surface configurations. For example, the substrate can be a flat glass substrate, such as a conventional microscope glass slide, a cover slip, and the like. Common substrates used for probe arrays are surface-derivatized glass or silica, gold, platinum, polyacrylate gels, or polymer membrane surfaces.

Kits for use in analyte detection assays also are contemplated in the present invention. The subject kits at least include the Morpholino probe arrays of the subject invention. The kits may further include one or more additional components necessary for carrying out the analyte detection assay, such as sample preparation reagents, buffers, labels, and the like. As such, the kits may include one or more containers such as vials or bottles, with each container containing a separate component for the assay, such as an array, and reagents for carrying out nucleic acid hybridization assays according to the invention. The kit may also contain microelectronic technology required to read out the assay results. Thus, the kit will comprise in packaged combination, a Morpholino probe array according to the subject invention, wherein the array comprises hybridization probes that sequence-selectively and detectably hybridize to target nucleotide sequences, and which may also comprise control probes included for data normalization and processing purposes.

### EXAMPLES

Morpholino probe films, and for comparison DNA probe films, on gold electrode supports were prepared using methods adapted from the literature. In a first step, both Morpholino probe strands and DNA probe strands modified with sulfhydryl or disulfide terminal groups were chemisorbed to the gold support, with the terminal groups reacting to form thiolate bonds with the gold. In addition to this end-specific attachment, the affinity of the nucleic bases for gold causes adsorption of the probes through the base sites. This surface adsorption interferes with the ability to undergo facile hybridization with target species. Therefore, in a second step, the nonspecific backbone-surface interactions were displaced by exposing the probe layer to a solution of an alkanethiol. The sulfhydryl moiety on the alkanethiol preferentially chemisorbs to the gold to displace the weaker base-gold interactions and to passivate, or block, the gold surface against further adsorption.

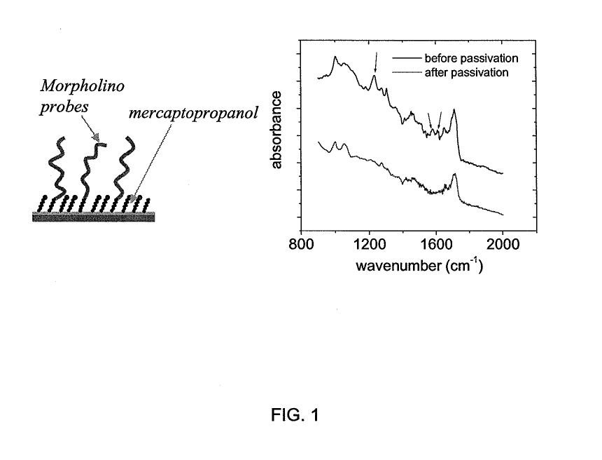

Mercaptopropanol (MCP) was used as the surface blocking agent in these examples, but other alkanethiols can be employed. The outcome leaves the probe chains in an end-tethered geometry, as shown in FIG. 1 (left).

Disulfide terminated Morpholino molecules were provided by Gene Tools, LLC. Solutions were prepared following manufacturer recommended protocol in deionized water, and the Morpholinos were allowed to adsorb to gold coated slides from concentrations ranging from 0.1 to 0.5 μM. FIG. 1 (right) shows infrared reflection-absorption (IRRAS; 4 cm⁻¹ resolution) spectra measured before (top line) and after (bottom line) treatment of the Morpholino modified gold with MCP. A 1 mM solution of MCP in deionized water was applied for 150 minutes. A thymine-rich Morpholino sequence was used because the thymine base is known to exhibit strong spectral features indicative of direct contact with gold. As shown by the arrows, spectral features characteristic of the base-gold contact disappear after treatment with MCP, confirming that adsorption of Morpholino bases to the gold was effectively disrupted. The “noise” in the after trace is due to atmospheric moisture. This same protocol was used to prepare Morpholino probe films used in subsequent examples, as described below.

Most of the data for the following examples was obtained in sodium phosphate buffer, at pH 7, room temperature (˜23° C.), and various strengths between 10 and 1000 mM in phosphate groups depending on the need of a particular measurement. The conditions used in each case are stated in the preceding Brief Description of the Drawings section. The type of buffer, however, is not of consequence and any of the known buffers suitable for hybridization assays and within the knowledge of those of ordinary skill in the art should work similarly (for example, citrate or tris buffers, as well as other standard biological buffers).

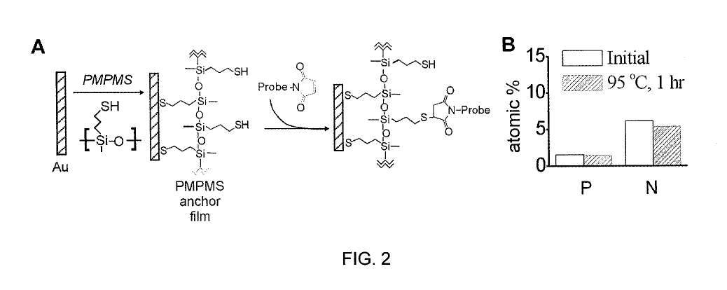

The above thiolate-based surface immobilization chemistry is stable up to temperatures moderately exceeding room temperature (for example, 45° C.), and good stability has been reported at temperatures up to about 60° C. when applied intermittently. However, at higher temperatures, loss of probes from the solid support occurs. For example, nearly complete removal of DNA probes was found after a 1 hour exposure to 95° C. buffer. As diagnostic assays are likely to require use of elevated temperatures (for example, to improve sequence stringency or to help denature target strands), it is important to use immobilization chemistry that is not subject to such limitations. Therefore, a robust tethering method was designed for high temperature applications based on polythiol immobilization. In this approach an “anchor” film of poly(mercaptopropyl) methylsiloxane (PMPMS) polymer, approximately 1 to 3 nm thick, is first formed on the gold support, followed by conjugation of maleimide-, acrydite-, disulfide-, or thiol-modified probes to available thiols of the anchor film. PMPMS can be deposited onto the solid support directly from the pure melt state, or by dipping or coating of the support from solutions in organic solvents such as toluene. Subsequent covalent attachment of Morpholino probes can be carried out from deionized water, following known methods in the art for these types of bioconjugate chemistries. FIG. 2A illustrates such surface conjugation using maleimide-modified DNA probes. With this immobilization, over 90% of DNA probes remained on the support after a 1 hour exposure to 95° C. buffer. This is shown in FIG. 2B in terms of the atomic percentages of phosphorus (P 2p) and nitrogen (N 1s), determined with X-ray photoelectron spectroscopy, before and after immersion of the probe layer in hot buffer. Both P and N elements are indicative of the immobilized DNA probe.

More generally, polythiols such as PMPMS can be used to complex metals, especially noble metals such as gold, through numerous thiolate bonds, rendering their attachment effectively irreversible. Probe molecules can then be coupled to remnant thiol groups of the polymer through various chemistries, such as those indicated above.

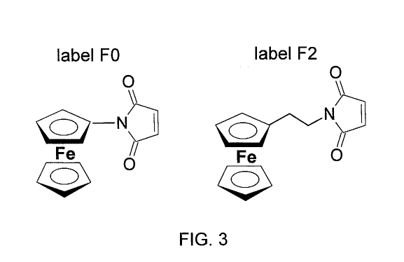

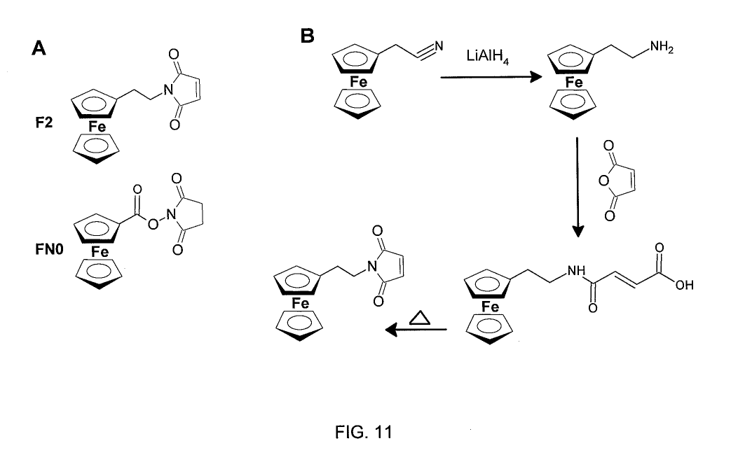

In order to quantitatively validate surface hybridization between Morpholino probes (immobilized on a solid support) and nucleic acid targets (present in solution), several electroactive tags for labeling of target and/or probe molecules were synthesized. These tags are capable of undergoing reversible cycling of their oxidation state. By measuring the total charge needed to convert the oxidation state of tags on targets or probes, the number of tags, and hence target or probe molecules, at the surface can be quantified. The tags contain an electroactive ferrocene moiety to provide the redox signal and a maleimide, NHS-ester, or acrydite group to allow conjugation to sulfhydryl or amine groups on target or probe molecules. The chemical structures of three such tags, ferrocene maleimide (F0) and ferrocene ethylmaleimide (F2), as well as N-hydroxysuccinimide ester of ferrocene carboxylic acid (FN0), are shown in FIGS. 3 and 11A. FN0's NHS ester is reactive toward amine groups, while F0's and F2's maleimide groups allow conjugation to sulfhydryl or amine groups. FIG. 11B outlines the chemical steps employed to synthesize the F2 tag from commercially available precursors. The synthesis of FN0 was based on carbodiimide-mediated coupling of the precursor ferrocene carboxylic acid and N-hydroxysuccinimide. The tag products were purified by solvent extraction and flash chromatography and their identity and purity were confirmed by NMR, IR, and visible spectroscopies. The tags are used to label target and probe strands so that probe and target surface coverages, and hence extents of hybridization, can be simultaneously monitored in-situ and in real time. This capability assists quantitative comparison of the relative performance of DNA and Morpholino probe assays in surface hybridization applications.

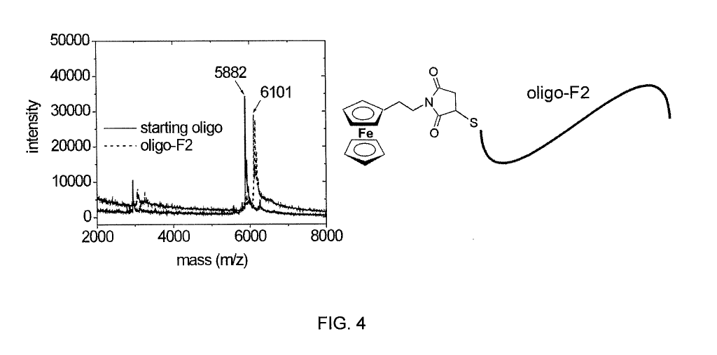

Protocols have been established for modification of nucleic acids and Morpholinos with the different ferrocene tags. For example, FIG. 4 shows the expected increase in molecular weight by 218 Daltons, representing addition of the F2 label (309 Daltons) and subtraction of a S-(CH₂)₃-OH fragment (91 Daltons) due to cleavage of the disulfide on the starting target nucleic acid. The additional peaks, displaced by about +40 Daltons, are attributed to various extents of complexation of potassium ions from the buffer with the target strands. Also shown is a schematic structure of the oligo-F2 conjugate. A Morpholino probe can be modified with these tags by dissolving the probe in deionized water at a concentration of 10 μM, and adding the tag to an approximately 100:1 molar excess over the Morpholino. If the tag is not sufficiently soluble in water, polar organic solvents such as acetonitrile can be used to dissolve the tag, followed by addition of the tag solution to the Morpholino solution in water. After 2 to 15 hrs of reaction, unreacted tags are removed by passing the solution over a size exclusion column, followed by collection of the modified Morpholino probe and further purification by high pressure liquid chromatography (HPLC) at 0.5 ml/min flowrate, and using a linear gradient of 12 to 100% methanol in solvent A over 30 minutes, where solvent A is 8.6 mM triethylammonium and 100 mM hexafluoroisopropyl alcohol in pH 8.1 water.

Results were obtained to demonstrate electrochemical monitoring of the hybridization of labeled targets, prepared as summarized above, with Morpholino and DNA probe films. Probe films of either Morpholino or DNA, 20 bases in length, were prepared on gold working electrodes and exposed to solutions of labeled complementary targets under various buffer conditions. Cyclic voltammetry (CV) measurements were carried out on the hybridized surfaces. For example, for a probe length of 20 bases, assay conditions could utilize Morpholino probe coverage of 5×10¹¹ probes/cm², ionic strength of 1 mM, and temperature of 35° C. Depending on probe and target lengths, embodiments of representative Morpholino probe assays can have a probe coverage of between about 1×10¹¹ to 2×10¹³ probes/cm², an ionic strength of between about 0.01 to 1000 mM, and be at a temperature of between about 20 to 70° C.

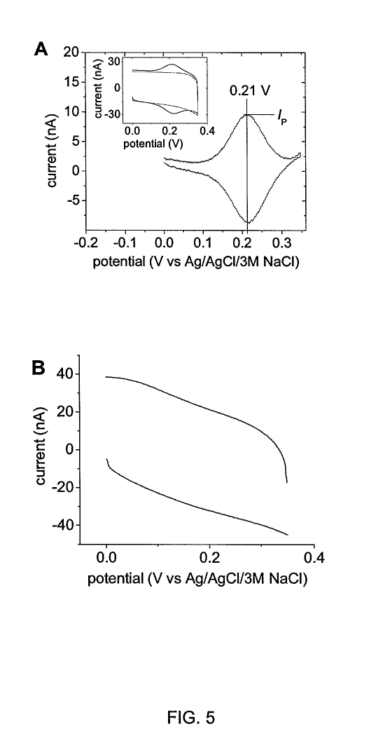

FIG. 5A inset shows a CV trace of a Morpholino probe layer in buffer before hybridization (bottom line) and after hybridization (top line) to complementary F0-labeled DNA target. The main panel shows the corresponding background-corrected signal (=hybridized scan minus buffer scan shown in inset). FIG. 5B shows a CV trace from a Morpholino probe film after contact with a solution of a noncomplementary F0-labeled target. The depicted hybridizations were carried out for 32 minutes in 1M phosphate buffer solution, pH 7, using 143 nM target concentration.

From the near equivalence of the peak positions (˜0.21 V) in FIG. 5A on the anodic (forward) and cathodic (reverse) sweeps, it is evident that the measured signal is from target strands that are immobilized at the surface, as opposed to from target strands diffusing to the electrode from solution (in which case a ˜60 mV splitting in the peak positions is expected). Moreover, plots of the peak current I_{P} versus the voltage sweep rate dV/dt revealed a linear dependence I_{P}˜dV/dt, a further confirmation that the signal is from surface-bound rather than from solution target species (for diffusing electroactive species, I_{P}˜(dV/dt)^{1/2} is expected). These results confirm that the labeled targets underwent immobilization at the surface, and the fact that noncomplementary sequences did not yield a clear diagnostic signal, as shown in FIG. 5B, indicates that the observed signals are indeed due to sequence-specific binding of nucleic acid targets to Morpholino probes. These results demonstrate that films of immobilized Morpholino molecules readily undergo hybridization with nucleic acid targets.

The surface coverage of hybridized targets can be calculated from the CV traces after subtraction of the charging background (FIG. 5A). The coverage is calculated by integrating the area under the corresponding/vs t curves, after converting the potential axis to time, to obtain the total charge passed. Each label provides 1 electron=1.6×10⁻¹⁹ Coulombs of charge. For example, for the data in FIG. 5A the coverage of probe-target hybrids is found to be 2.7×10¹² cm⁻².

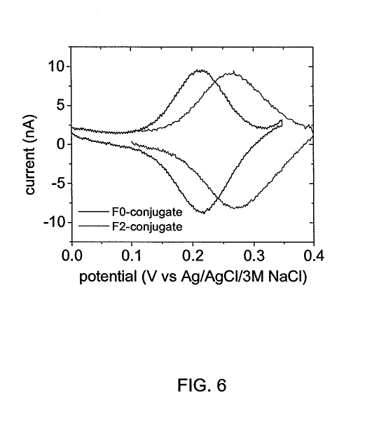

The oxidation potentials of F2 and F0 labeled targets, when hybridized to Morpholino films at comparable coverages, are separated by about 60 mV (FIG. 6). The difference in oxidation potentials affords, in principle, the capability to simultaneously monitor surface populations of two different target species (for example, “dual color” electrochemical assays). The exact peak positions depend on the local environment, as discussed further below.

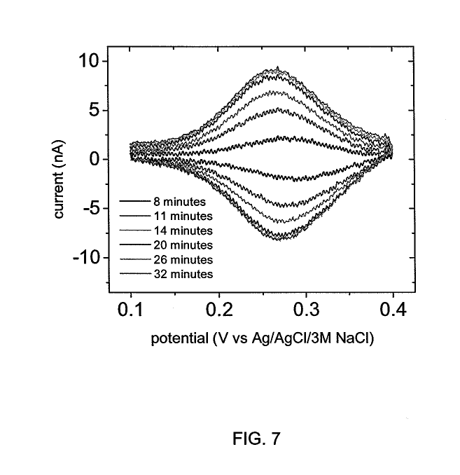

Electrochemical measurements can be carried out as a function of time while hybridization is progressing to monitor the coverage of bound target species. The data in FIG. 7 demonstrate real-time monitoring of hybridization for a Morpholino probe layer undergoing hybridization to complementary, F2-labeled target at 140 nM concentration in 1M phosphate buffer, pH 7. The reaction approaches completion after 20 minutes when a 140 nM target concentration is used. From FIG. 7 one can see that the oxidation potential of the tag shifts negatively by about 20 mV as hybridization proceeds. This phenomenon reflects increased stabilization of the positively charged ferricinium state of the tag due to accumulation of negative charge in the probe layer from the hybridized DNA targets.

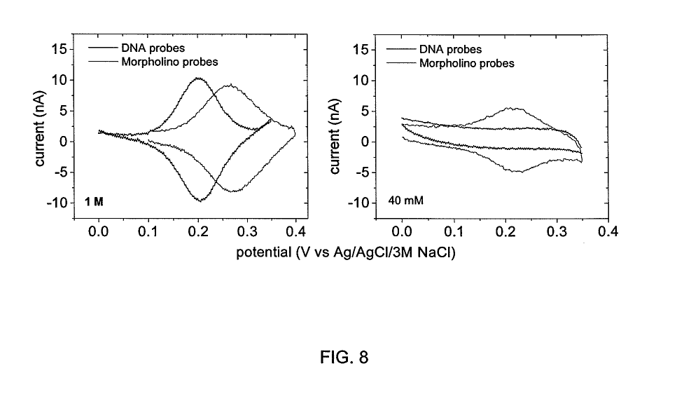

Results also have been obtained to establish that Morpholino probe films, in contrast to DNA probe films, can be used under assay conditions of low ionic strength. FIG. 8 compares hybridization signals measured after hybridization to complementary target under 1M (FIG. 8 left) and 40 mM (FIG. 8 right) phosphate buffer ionic strengths and otherwise identical conditions. Background charging currents have been subtracted. The hybridization signals were confirmed to saturate as in FIG. 7, indicating completion of the hybridization reaction. At 1M ionic strength, similar signals are obtained using both types of probe, confirming approximately comparable activity toward target species. In contrast, under 40 mM ionic strength the Morpholino assay exhibits a much stronger signal. This behavior is qualitatively expected; at low ionic strengths the highly negatively charged layer of DNA probes will repel the negatively charged target species, suppressing their hybridization. Interestingly, although the effect is less than for DNA probes, hybridization of targets to the Morpholino probe film is also suppressed at 40 mM relative to the 1M condition. This suppression reflects accumulation of electrostatic penalty to hybridization from already hybridized targets. Given a complex target background with many target sequences, the electrostatic penalty is expected to increase the stringency (sequence fidelity) of surface hybridization as an assay progresses, leading to a melt off of target species that are only partially complementary to the probes.

The capability of the Morpholino probe assays to perform surface hybridization at reduced ionic strengths is central as it is under such conditions that unique benefits (for example, disruption of secondary structure in target species, no need for a washing step) of the Morpholino probe technology are best realizable. The preferred experimental conditions for carrying out surface hybridization of Morpholino probes to nucleic acid targets can vary over a range of ionic strength, from less than 1 mM to 1M. Other experimental conditions fall within ranges similar to those used with surface hybridization of DNA probes.

It is interesting to note the difference in the CV trace peak position for the Morpholino and DNA probe films in FIG. 8 (left). In both cases, the targets were tagged with the same F2 label. Thus, the shift in position of the redox peaks must derive from the influence of the local environment on the oxidation of the F2 label. The data reveal that the oxidation F2→F2⁺+e⁻ is easier in the DNA probe layer, where it tends to occur at the lower potential of about 0.21 V, compared to ˜0.27 V for the Morpholino probe layer. Evidently, the presence of negative DNA probe charge stabilizes the positive F2⁺ oxidation state. The sensitivity of the peak potential to the type of probe underscores the importance of probe charge in contrasting the DNA and Morpholino probe systems. The shift in redox potential does not affect quantification of target strands since the stoichiometry of one electron per tag is not affected by the peak shift.

Morpholino probes should be especially well adapted to label-free detection methods based on electrostatic principles and interfacial charge organization. One such approach involves measurement of interfacial capacitance. Capacitance represents the ability of the solid-liquid interface to store charge. If the local environment at the solid-liquid interface becomes more capable of screening electrostatic interactions, then the interfacial capacitance increases because more charge must be added to the interface to realize a given potential difference across it. Examples of physical changes that can lead to an increased capacitance include an accumulation of charge carriers at the interface (e.g., a higher ionic strength) and an increase in the local dielectric constant. Opposite changes in these quantities would lead to a lowered interfacial capacitance.

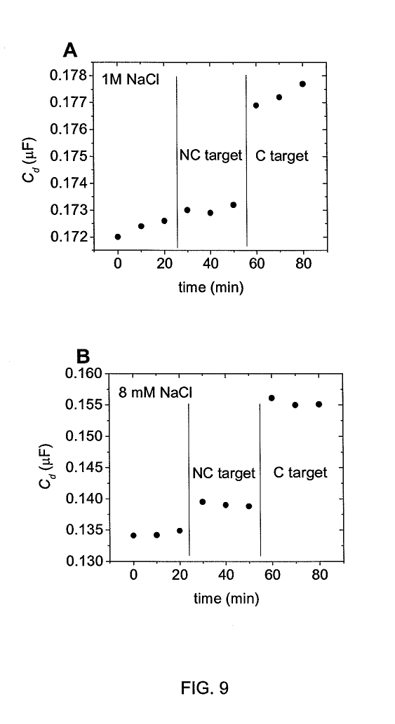

FIG. 9 shows results in which capacitance of 20mer Morpholino probe films was monitored as a function of time while in contact with 140 nM solutions of 18mer targets in NaCl solutions, pH 7. The electrochemical technique of electrochemical impedance spectroscopy was used to determine the differential surface capacitance. Morpholino probe films were first exposed to a solution of a noncomplementary (NC) sequence followed by a subsequent addition of the complementary (C) sequence to make a mixed NC/C solution. Vertical lines separate these different stages of the assay. FIG. 9A was obtained under 1M ionic strength, while FIG. 9B was measured under 8 mM NaCl. In both cases, some changes are apparent after addition of the NC sequence, with significantly stronger responses observed following addition of the C sequence.

Several observations are noteworthy. First, the data demonstrate that hybridization between Morpholino probes and complementary nucleic acid targets can be clearly resolved from changes in interfacial capacitance. Second, under 8 mM salt, the response to the complementary sequence C is much stronger (˜11% increase in capacitance) compared to 1M salt (˜2% increase in capacitance). This observation agrees with the qualitative expectation that lower salt concentrations will enhance electrostatic effects due to probe-target binding. In the case considered, the greater response at low salt may reflect changes in the local ionic strength brought by hybridization. Third, at the lower salt a nonspecific signal is seen from the NC addition. This may be due to image charge attractions between the NC target strands and the highly polarizable metal underlayer. At 8 mM conditions, the electrostatic screening length is about 3.3 nm. Therefore, image charge attractions would be expected to act over several nm. Thus, if the probe layer allows NC targets to approach to within a few nm of the electrode, the targets may be expected to adsorb due to image charge interactions. If so, application of repulsive electrostatic potentials will be an effective means to decrease binding of NC.

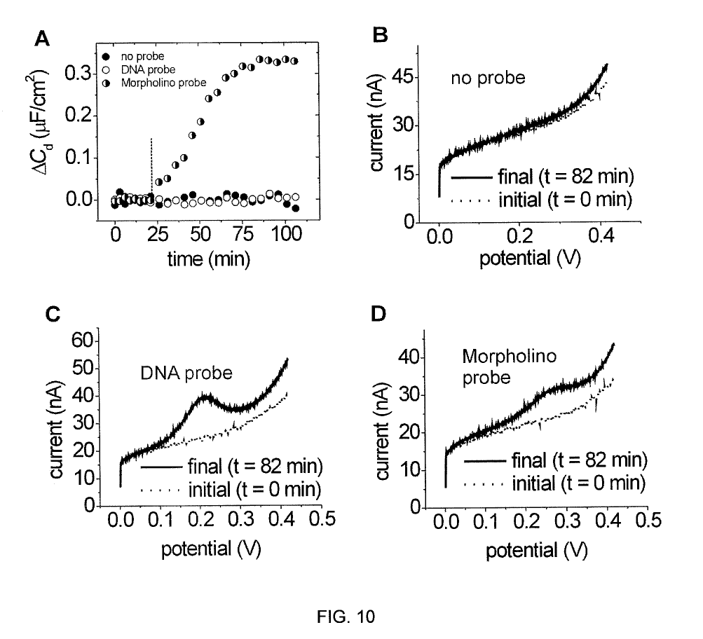

Capacitive detection is a label-free, real-time sensing method that monitors the change in capacitance (i.e. in the current response to an imposed change in surface potential) of the probe layer as a function of its hybridization state. The data in FIG. 10 show that DNA probe layers can exhibit little, if any, capacitive response to binding of target strands (FIG. 10A open circles). In fact, the capacitive response can be very similar to controls that do not undergo hybridization, i.e. when only the blocking MCP layer is present on the surface as in FIG. 10A, black dots. Because the targets were labeled with the tag F2, simultaneous CV measurements could be taken to confirm that hybridization did, indeed, take place on the DNA probe layer (FIG. 10C). The lack of capacitive signal in the case of the DNA probe layer was attributed to the high background charge of the DNA probe layer itself, resulting in decreased sensitivity to subsequent addition of target charge during hybridization. In these measurements, the surface was maintained at the open circuit potential (OCP: about 75 mV vs Ag/AgCl/3M NaCl) of the unhybridized layer, and a 1M phosphate buffer at pH 7 was used. Working at other conditions, e.g. other probe coverages, buffer strengths, or surface potentials, could possibly increase capacitive response of DNA probe assays; however, the lack of signal in FIG. 10A clearly suggests that the response would likely be weak. In striking contrast, uncharged Morpholino probe layers provided a strong increase in capacitance when hybridized with nucleic acid targets (FIG. 10A-medium circles). Dividing the total change in capacitance due to surface hybridization (0.32 μF/cm²) by the standard deviation of the values from the unhybridized layer (0.0033 μF/cm²) yields a 97:1 (˜40 dB) SNR ratio, corresponding to a target coverage of 2.1×10¹² cm⁻² as determined from the CV data of FIG. 10D. Assuming a linear response, a target coverage of 2.2×10¹¹ cm⁻² should therefore be detectable at an SNR of 20 dB. These results provide a compelling demonstration of the benefits of enhanced electrostatic selectivity and sensitivity (here demonstrated through surface capacitance measurements) to surface hybridization of target species when the probes are not charged, as in the case of Morpholino probes.

Although the data in FIGS. 9 and 10 were obtained for a geometry in which Morpholino probe films are fabricated on a planar, conductive solid support, similar effects would also be operative for other geometries and supports. For example, Morpholino probes could be used to modify nanowires, carbon nanotubes, or otherwise shaped and/or fabricated conductive or semiconductive structures. The unifying theme in all such situations is that an improved diagnostic performance is realized because of more detectable changes in the electrical charge organization of the probe layer due to hybridization with nucleic acid targets, when Morpholino probes, which are not charged, are used in the assay. These benefits are especially pronounced when Morpholino probes are used because of their good aqueous solubility, outstanding coupling yields during synthesis, and superior sequence-specificity in complex (that is, possessing a large pool of sequences) target samples over other uncharged probes such as peptide nucleic acids or methylphosphonate materials. In addition to using interfacial capacitance to detect probe-target binding, analogous electrostatic modalities of detection of hybridization between immobilized Morpholino probes and nucleic acid targets in solution also include measurements traditionally referred to as “field-effect” where the flow of current and/or the potential inside the solid support (for example, an electrode, a nanowire, a field-effect transistor, a nanotube, or a similar structure) is influenced by electrostatic interactions between the Morpholino-target layer and the electrons inside the solid support. In addition, electrostatic detection of surface hybridization between Morpholino probes and nucleic acid targets can be realized through transduction by the partitioning behavior of charged, electroactive species (for example: ferrocyanide, hexaamine ruthenium) into the Morpholino probe layer, which could be monitored with cyclic or square wave voltammetry, chronocoloumetry, faradaic impedance, AC voltammetry, or other electrochemical methods.

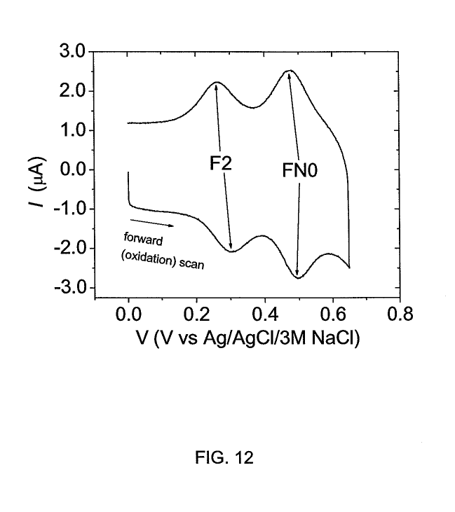

FIG. 12 shows a cyclic voltammogram (CV) trace of a Morpholino probe layer hybridized with complementary DNA target in 1 M phosphate buffer. These types of measurements were used to compare Morpholino and DNA surface hybridization behavior, discussed below. The Morpholino probes were labeled with the tag FN0, the targets with the tag F2. The separation of the probe and target tags is seen to be around 0.2V, with the F2 oxidation/reduction peak pair around 0.28 V and that for FN0 closer to 0.5V. The areas of the peaks can be integrated and converted to probe and target coverages as described earlier. Because of the increased capacitance C at positive potentials, the RC time constant at the onset of the reverse scan is larger and bleeds into the FN0 reduction peak. Therefore, coverages are best calculated from peak areas for the forward oxidation (anodic) wave. The target and probe coverages in FIG. 12 were measured to be approximately 4.8×10¹² cm².

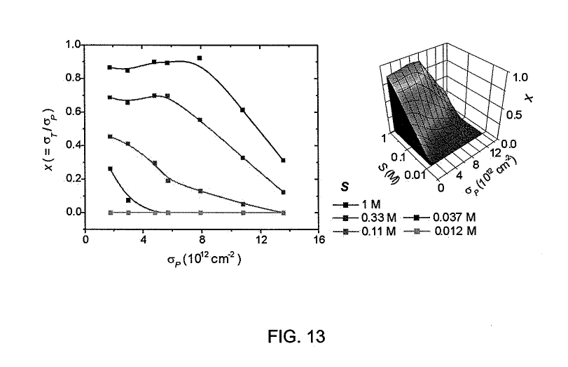

FIG. 13 shows data from a study of the equilibrium extents of surface hybridization between DNA targets and DNA probes as a function of probe coverage σ_{P} and salt concentration S, under noncompetitive conditions. These experiments used the tag labeling chemistries and measurements discussed above (e.g. FIG. 12) to determine target coverages. Several distinct regimes of hybridization were identified, including a non-hybridizing (NH) regime at high probe coverages and low ionic strengths and a suppressed hybridization SH regime within which the fraction of hybridized probes x (x=σ_{T}/σ_{P}; σ_{T}: target coverage) varied with the probe coverage. This dependence on probe coverage is a clear signature of non-Langmuirian behavior indicating that steric and/or electrostatic interactions between probe sites were influencing hybridization. The SH to NH transition, corresponding to the onset of hybridization, was defined by the condition that counterion concentration in the probe layer, S_{layer}, was comparable to that in solution, S. This condition is expected to apply as long as electrostatics dominate the SH to NH transition. At somewhat lower probe coverages, the defining characteristic of the pseudo-Langmuir (PL) regime was near independence of x on probe coverage, despite physical closeness of surface sites and thus compulsory presence of site-site interactions based on geometrical considerations. This independence suggests that the pliable nature of the probe layer moderates such interactions through a structural reorganization (e.g. through reorientation of probe-target hybrids) so as to mitigate repulsive site-site interactions. The independence of x on probe coverage makes the PL regime especially attractive for diagnostic applications. More significantly, the data of FIG. 13 show that DNA probe layers fail to hybridize with nucleic acid targets at low salt concentrations and higher probe coverages.

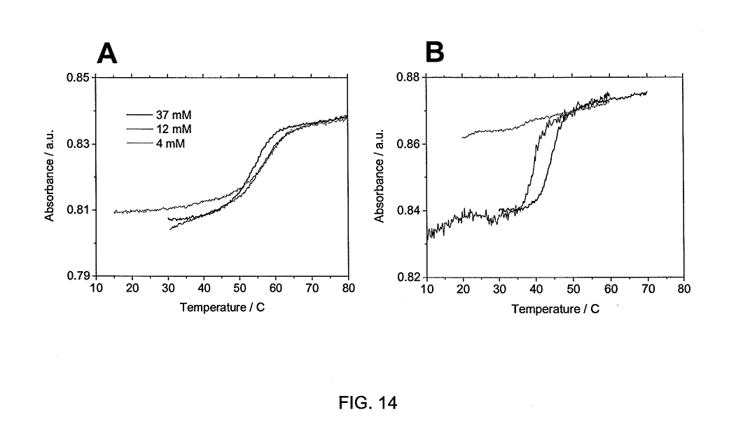

It is interesting to compare the above surface hybridization behavior of DNA probe surfaces with that of Morpholino probe surfaces, especially under low salt conditions under which benefits of denaturing of target-target associations (secondary structure), higher sequence stringency of the assay, and enhanced diagnostic sensitivity to binding of target strands (e.g. through capacitance or other label-free transduction) are anticipated. For example, in order to realize denaturing assays, it is necessary to operate under conditions for which target secondary structure is disrupted, while Morpholino-target hybridization is preserved. The melting curves in FIG. 14 demonstrate that stability of Morpholino-DNA hybrids is largely unaffected by low salt conditions, while that of double-stranded DNA hybrids is severely degraded. These melting curve results indicate that suitable hybridization conditions, for which nucleic acid sample would be significantly denatured but binding between Morpholino probes and nucleic acid targets would be preserved, are salt concentrations at or below 100 mM and temperatures of 30° C. or higher. These estimates also agree with available melting point correlations (e.g. for polymeric double-stranded DNA the melting point at 1 mM salt for a 100mer with 45% GC content is predicted to be 45° C.). In general, denaturing assays will require operation at or above the melting temperature of the solution nucleic acid sample, while simultaneously remaining at least 5° C. below the melting temperature of the surface-bound hybrids between Morpholino probes and complementary target molecules.

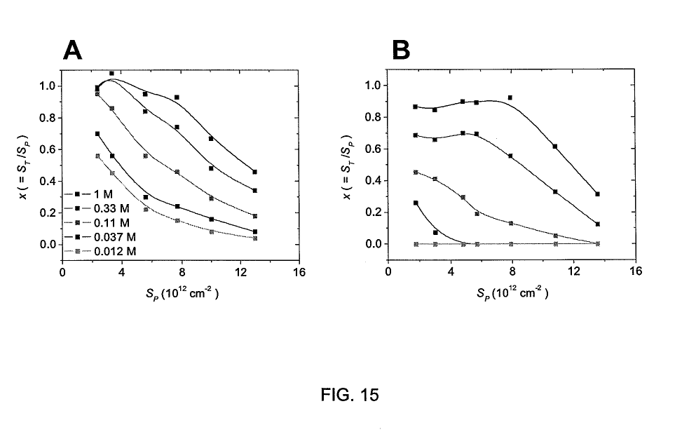

Hybridization activity on solid supports between Morpholino probes and solution DNA targets has been systematically screened. The results from 30 assays, for variable probe coverage and concentration of phosphate buffer (pH 7), are presented in FIG. 15A which plots the equilibrium extent of hybridization x=S_{T}/S_{P} (S_{T}: coverage of hybridized target; S_{P}: coverage of probe sites) as a function of salt concentration (0.012 to 1 M phosphate) and probe coverage. Strand surface coverage data were obtained using methods of analysis as described in connection with FIG. 12. The results of FIG. 15A are compared to those from DNA probe assays, under identical conditions and for the same sequences, in FIG. 15B. Key observations include: 1) surface hybridization assays with Morpholino probes exhibit higher binding affinities than with DNA probes, especially at lower salt concentrations and lower probe coverages. This higher affinity is essential to detection of target species at lower concentrations. 2) In agreement with solution studies (FIG. 14), Morpholino probe surface hybridization assays proceed under low salt conditions, where hybridization using DNA probes is impossible. This activity under low salt enables implementation of denaturing assays, as discussed earlier. 3) A comparison of surface (FIG. 15B) and known solution hybridization thermodynamics in the case of DNA probes implicated probe-probe interactions as significantly suppressing surface hybridization affinities (by 9 orders of magnitude for the experiments in FIG. 15B) toward complementary target strands. Similar considerations are expected to also apply to Morpholino probe assays; therefore, probe-target surface hybridization affinities should be enhanced by using probe layers in which the probes are sufficiently sparsely immobilized so as to not strongly interact with one another.

The above results triangulate low salt (100 mM or lower), moderate to high temperature (30° C. or higher), and low probe coverage (2×10¹² cm⁻² or lower) as conditions that simultaneously disrupt target secondary structure, avoid probe-probe associations, and provide for strong hybridization between Morpholino probes and nucleic acid targets. However, as precise settings depend on the probe length used for the assay, more generally conditions for Morpholino probe surface hybridization assays of the present invention should be optimized between 0.01 mM to 1000 mM ionic strength, 20° C. to 70° C. temperature, and 1×10¹¹ cm⁻² to 2×10¹³ cm² probe coverage.

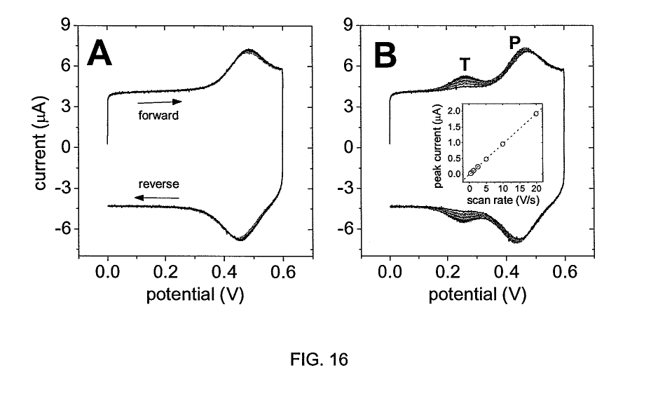

FIG. 16 depicts a hybridization series for a buffer strength of 200 mM phosphate buffer, pH 7, on a Morpholino probe layer blocked with MCP. At the outset, the probe layer was contacted with noncomplementary target labeled with F2 and CV data were obtained every 5 minutes for 50 minutes. In FIG. 16A data are plotted from ten such consecutive CV scans, when noncomplementary target was present at 25 nM. A Morpholino probe peak near 0.48 V is observed due to the probes FN0 tag. However, no electroactivity from target is evident, indicating that noncomplementary binding was below the detection limit of ˜1×10¹¹ targets/cm². If present, the target signal, from the F2 tag, would appear close to 0.25 V. These data also demonstrate the good stability of the Morpholino surface, with all ten curves closely superposing.

Following the above noncomplementary measurements, complementary target strands, also labeled with the F2 tag, were added to realize a 1:1 noncomplementary:complementary target mixture at 25 nM each. Hybridization commenced immediately when complementary target was added, manifesting in a peak at 0.25V (FIG. 16B). These data demonstrate that the Morpholino probe layer is able to discriminate the presence of the complementary target sequence from the mixture. The near equivalence of target oxidation and reduction peak potentials seen, respectively, on the forward and reverse scans at 0.25V confirms that the signal is from surface-bound targets and not from targets diffusing in solution. Further confirmation of the surface origins of the target signal was performed by verifying that the peak current, measured from baseline, scaled linearly with the scan rate d V/dt (inset to FIG. 16B).

FIG. 16A shows ten CV traces, obtained 5 minutes apart, from a Morpholino probe layer in contact with a 25 nM solution of noncomplementary target in 200 mM phosphate buffer solution at pH 7. FIG. 16B shows ten CV traces, obtained 5 minutes apart, from the same Morpholino probe film after introduction of 25 nM concentration of complementary target into the noncomplementary solution, to make a 1:1 noncomplementary-to-complementary target mixture. Other settings: scan rate=20 V/s; probe coverage=3.6×10¹² probes/cm²; probe sequence: 5′FN0-TTT TAA ATT CTG CAA GTG AT-S-S-R 3′; complementary target sequence: 5′F2-ATC ACT TGC AGA ATT TAA 3′; noncomplementary target sequence: 5′ AAA AAA AGG MG GM AAA-F2 3′.

The above description sets forth the best mode of the invention as known to the inventor at this time, and is for illustrative purposes only, as it is obvious to one skilled in the art to make modifications to this process without departing from the spirit and scope of the invention and its equivalents as set forth in the appended claims.

What is claimed is:

1. A method for obtaining sequence and/or concentration information of nucleic acid molecules, comprising the steps of:
a) providing surface-immobilized nonionic Morpholino molecules; and
b) initiating a surface-based hybridization between the Morpholino molecules and the nucleic acid molecules in a solution,
wherein associations between the nucleic acid molecules are disrupted and associations between the Morpholino molecules and the nucleic acid molecules are preserved.

2. The method as claimed in claim 1, wherein the hybridization is conducted at combinations of ionic strength of the solution and temperature of the solution are such that the associations between the nucleic acid molecules are disrupted and the associations between the Morpholino molecules and the nucleic acid molecules are preserved, whereby the disrupted associations between the nucleic acid molecules do not interfere with a hybridization to the surface-immobilized Morpholino molecules.

3. The method as claimed in claim 2, wherein the Morpholino molecules are covalently bonded to a substrate surface to form a Morpholino probe.

4. The method as claimed in claim 3, wherein the substrate is selected from the group consisting of gels, sheets, tubing, spheres, containers, pads, slices, films, plates, slides, strips, plates, disks, rods, particles, microelectronic chips, and beads.

5. The method as claimed in claim 4, wherein the immobilization surface is a material selected from the group consisting of surface-derivatized glass, gold, silicon oxide, polyimide, silicon nitride, and polymer and metal material surfaces.

6. The method as claimed in claim 3, wherein the Morpholino molecules are covalently bonded to the substrate surface by a tethering method comprising the steps of:
forming an anchor film of poly(mercaptopropyl) methylsiloxane (PMPMS) polymer, approximately 1 to 3 nm thick, on the substrate surface; and
conjugating maleimide-, acrydite-, disulfide-, or thiol-modified Morpholino molecules to available thiols of the anchor film.

7. The method as claimed in claim 3, wherein the Morpholino probe has a probe coverage of between about 1×10¹¹ to 2×10¹³ probes/cm², the solution has an ionic strength of between about 0.01 to 1000 mM, and the solution is at a temperature of between about 20 to 70° C.

8. The method as claimed in claim 2, wherein the associations between the nucleic acid molecules are disrupted such that they do not interfere with the associations between the Morpholino molecules and the nucleic acid molecules.

9. A method for obtaining sequence and/or concentration information of nucleic acid molecules, comprising the steps of:
a) providing surface-immobilized nonionic Morpholino molecules covalently bonded to a substrate surface to form a Morpholino probe, the Morpholino molecules being covalently bonded to the substrate surface by a tethering method comprising forming an anchor film of poly(mercaptopropyl) methylsiloxane (PMPMS) polymer, approximately 1 to 3 nm thick, on the substrate surface and conjugating maleimide-, acrydite-, disulfide-, or thiol-modified Morpholino molecules to available thiols of the anchor film; and
b) initiating a surface-based hybridization between the Morpholino molecules and the nucleic acid molecules in a solution,
wherein the hybridization is conducted at combinations of ionic strength of the solution and temperature of the solution such that the associations between the nucleic acid molecules are disrupted and the associations between the Morpholino molecules and the nucleic acid molecules are preserved.

10. The method as claimed in claim 9, wherein the Morpholino probe has a probe coverage of between about 1×10¹¹ to 2×10¹³ probes/cm², the solution has an ionic strength of between about 0.01 to 1000 mM, and the solution is at a temperature of between about 20 to 70° C.

11. The method as claimed in claim 10, wherein the substrate is selected from the group consisting of gels, sheets, tubing, spheres, containers, pads, slices, films, plates, slides, strips, plates, disks, rods, particles, microelectronic chips, and beads.

12. The method as claimed in claim 11, wherein the immobilization surface is a material selected from the group consisting of surface-derivatized glass, gold, silicon oxide, polyimide, silicon nitride, and polymer and metal material surfaces.

13. A Morpholino probe assay for obtaining sequence and/or concentration information of nucleic acid molecules, comprising surface-immobilized nonionic Morpholino molecules, wherein the Morpholino molecules are covalently bonded to a substrate surface to form the Morpholino probe.

14. The Morpholino probe assay as claimed in claim 13, wherein the substrate is selected from the group consisting of gels, sheets, tubing, spheres, containers, pads, slices, films, plates, slides, strips, plates, disks, rods, particles, microelectronic chips, and beads.

15. The Morpholino probe assay as claimed in claim 14, wherein the substrate is a material selected from the group consisting of surface-derivatized glass, gold, silicon oxide, polyimide, silicon nitride, and polymer and metal material surfaces.

16. The Morpholino probe assay as claimed in claim 13, wherein the Morpholino molecules are covalently bonded to the substrate surface by a tethering method comprising the steps of:
forming an anchor film of poly(mercaptopropyl) methylsiloxane (PMPMS) polymer, approximately 1 to 3 nm thick, on the substrate surface; and
conjugating maleimide-, acrydite-, disulfide-, or thiol-modified Morpholino molecules to available thiols of the anchor film.

17. The Morpholino probe assay as claimed in claim 13, wherein the Morpholino probe has a probe coverage of between about 1×10¹¹ to 2×10¹³ probes/cm², the solution has an ionic strength of between about 0.01 to 1000 mM, and the solution is at a temperature of between about 20 to 70° C.


SEQUENCE LISTING (11 sequences)

SEQ ID NO: 1 - DNA, 20 bp, Artificial Sequence
  Description of Artificial Sequence Synthetic probe
tttttttccttccttttttt

SEQ ID NO: 2 - DNA, 20 bp, Artificial Sequence
  Description of Artificial Sequence Synthetic probe
ttttaaattctgcaagtgat

SEQ ID NO: 3 - DNA, 18 bp, Artificial Sequence
  Description of Artificial Sequence Synthetic probe
atcacttgcagaatttaa

SEQ ID NO: 4 - DNA, 18 bp, Artificial Sequence
  Description of Artificial Sequence Synthetic probe
aaaaaaaggaaggaaaaa

SEQ ID NO: 5 - DNA, 18 bp, Artificial Sequence
  Description of Artificial Sequence Synthetic probe
atcacttgcagaatttaa

SEQ ID NO: 6 - DNA, 18 bp, Artificial Sequence
  Description of Artificial Sequence Synthetic probe
atcacttgcagaatttaa

SEQ ID NO: 7 - DNA, 18 bp, Artificial Sequence
  Description of Artificial Sequence Synthetic probe
aaaaaaaggaaggaaaaa

SEQ ID NO: 8 - DNA, 20 bp, Artificial Sequence
  Description of Artificial Sequence Synthetic probe
ttttaaattctgcaagtgat

SEQ ID NO: 9 - DNA, 20 bp, Artificial Sequence
  Description of Artificial Sequence Synthetic probe
ttttaaattctgcaagtgat

SEQ ID NO: 10 - DNA, 18 bp, Artificial Sequence
  Description of Artificial Sequence Synthetic probe
atcacttgcagaatttaa

SEQ ID NO: 11 - DNA, 18 bp, Artificial Sequence
  Description of Artificial Sequence Synthetic probe
aaaaaaaggaaggaaaaa